(19) United States
(12) Patent Application Publication
(10) Pub. No.: US 2004/0191193 A1
(43) Pub. Date: Sep. 30, 2004

(54) Method of administering active agents into the nail

(76) Inventors: Carol Collins, Granada Hills, CA (US); Lewis Bealer, Santa Monica, CA (US); Yohini Appa, (US); Sidney Hornby, San Pedro, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/401,470

(22) Filed: Mar. 28, 2003

(51) Int. Cl. A61K 7/04

(52) U.S. Cl. 424/61

(57) ABSTRACT

The present invention relates to a composition suitable for application as a nail polish containing (i) an active agent, (ii) at least one film-forming polymer, and (iii) a carrier comprising water, alcohol, or a mixture thereof, wherein the composition has a percent film retention at 30 minutes of from about 95% to about 50%.

### FIELD OF THE INVENTION

The present invention relates to a composition for application to the nail.

### BACKGROUND OF THE INVENTION

Nail polishes typically contain harsh chemicals and solvents such as butyl acetate, ethyl acetate, nitrocellulose, toluene sulphonamide formaldehyde resin, dibutyl phthgalate, toluene, styrene, and acetone. These chemicals can cause the surface of the nail to become dry and brittle. In addition, an aggressive solvent, such as acetone, is often required to remove the polish from the nail. This process can further damage the nail.

Thus, it is desirable for a product that will be provide shine, color, and/or conditioning to the nail, but contain less amounts of harsh chemicals and/or does not require aggressive organic solvents for removal.

### SUMMARY OF THE INVENTION

The present invention relates to a composition suitable for application as a nail polish containing (i) at least one film-forming polymer and (ii) a carrier containing water, alcohol, or a mixture thereof, wherein the composition has a percent film retention at 30 minutes of from about 95% to about 50%.

The present invention further relates to a method of promoting a composition suitable for application to the nail by instructing the user of the composition (i) to apply the composition to the nail; and (ii) to remove the composition from the nail by washing with water; wherein the composition has a percent film retention at 30 minutes of from about 95% to about 50%.

The present invention also relates to a method for administering an active agent into the nail by applying to the nail a composition containing (i) an active agent, (ii) at least one film-forming polymer and (iii) a carrier containing water, alcohol, or a mixture thereof, wherein the composition has a percent film retention at 30 minutes of from about 95% to about 50%.

The present invention further relates to a method for strengthening or conditioning the nail by applying to the nail a composition containing (i) panthenol, (ii) at least one film-forming polymer, and (iii) a carrier comprising water, alcohol, or a mixture thereof, wherein the composition has a percent film retention at 30 minutes of from about 95% to about 50%.

Other features and advantages of the present invention will be apparent from the detailed description of the invention and from the claims

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

Definitions

What is meant by a “product” is a product in finished packaged form. In one embodiment, the package is a container such as a plastic, metal or glass tube or jar containing the composition. The container may further include an applicator to topically apply the composition to the mammalian nail such as a foam or bristle brush. The product may further contain additional packaging such as a plastic or cardboard box for storing such container. In one embodiment, the product contains instructions directing the purchaser and/or user to apply the composition to the nail (e.g., to provide shine, color, and/or therapeutic or prophylactic benefit to the nail). The instructions may also contain instructions to remove the composition from the nail by washing with water. What is meant by “washing with water” is rubbing the nail containing the composition (e.g., with fingers or a substrate such as a cloth) with water. In one embodiment, detergents such a soap may also be used during the washing. In one embodiment, the product also contains instructions that harsh solvents such as acetone are not needed to remove the composition from the nail. Such instructions may be printed on the container, label insert, or on any additional packaging.

What is meant by “promoting” is promoting, advertising, or marketing. Examples of promoting include, but are not limited to, written, visual, or verbal statements made on the product or in stores, magazines, newspaper, radio, television, internet, and the like. Examples of such statements include, but are not limited to: add shine to the nail; add color to the nail; strengthen the nail; reduce breakage, chipping, and/or peeling of the nail; condition the nail; remove the composition by washing with water; no need to use harsh solvents to remove the composition from the nail; and contains no harsh solvents and/or chemicals.

As used herein, “topically applying” means directly laying on or spreading on the nail, e.g., by use of an applicator included with the product.

As used herein, “cosmetically-acceptable” means that the ingredients which the term describes are suitable for use in contact with tissues (e.g., the skin or nail) without undue toxicity, incompatibility, instability, irritation, allergic response, and the like.

As used herein, “safe and effective amount” means an amount of the ingredient or of the composition sufficient to provide the desired benefit at a desired level, but low enough to avoid serious side effects. The safe and effective amount of the ingredient or composition will vary with the area being treated, the age of the end user, the duration and nature of the treatment, the specific ingredient or composition employed, the particular carrier utilized, and like factors.

As used herein, the term “nail polish” is a comprehensive term describing a nail polish composition, film, product (including coloring products), system, kit, or the like, which is useful for providing, for example, aesthetic, therapeutic, or prophylactic benefits to the nail.

As used herein, the term “mammalian nail” means a keratinaceous plate present at the upper surface of the end of a finger or toe of a primate, most preferably a human, or the homologous claw or hoof of another mammal.

Film-Forming Polymer

The compositions of the present invention contain at least one film-forming polymer. As used herein, the term “film-forming polymer” means a homopolymer, copolymer, or mixture thereof which forms an adherent continuum from a composition when applied to mammalian nails. As used herein, the term “copolymer” includes linear, block, branched, graft, comb, and star copolymers. The polymerization of the polymer may not actually take place until application of the composition is performed. Thus, the term “film-forming polymer” is meant to encompass monomers that have not yet polymerized but will upon application to the nail.

The film-forming polymers herein can be selected from nonionic, ionic (anionic or cationic), and amphoteric (including zwitterionic) polymers.

In one embodiment, the film-forming polymers herein are solvent-borne or water-borne polymers. As used herein, the term “water-borne”, with reference to a film-forming polymer, means that the polymer was prepared in a mixture containing water and is preferably added to the composition as a mixture (such as a dispersion) in water. As used herein, the term “solvent-borne”, with reference to a film-forming polymer, means that the polymer was prepared under substantially anhydrous conditions and is preferably added to the composition as a substantially anhydrous mixture (such as a solution).

Preferred film-forming polymers of the present invention are selected from polyacryls, polymethacrylics, polyurethanes, polyvinyls, and polysilicone-polyacryls polymers. The term “polyacryl” includes, but is not limited to, polyacrylates, polyacrylics, and polyacrylamides. The term “polymethacryl” includes, but is not limited to, polymethacrylates, polymethacrylics, and polymethacrylamides. The term “polysilicone-polyacryls” includes, but is not limited to, polysilicone-8, polysilicone-11, polysilicone-6, Polysilicone-14, Isobutylmethacrylate/Bis-Hydroxypropyl Dimethicone Acrylate Copolymer. Examples of preferred film-forming polymers include, but are not limited to, acrylates copolymer, such Avalure AC315 (Noveon, Cleveland, Ohio USA), and polysilicone-8, such as Silicone Plus Polymer VS80 (3M, St. Paul, Minn. USA).

In one embodiment, the composition includes from about 1 to about 50 percent, by weight, of at least one film forming polymer, such as from about 5 to about 20 percent, by weight, of at least one film forming polymer.

Carrier

The compositions of the present invention further contain a carrier that includes water and/or an alcohol. Examples of alcohols include, but are not limited to, ethanol, methanol, isopropanol, and butanol. In one embodiment, the composition does not contain any harsh chemicals and solvents such as butyl acetate, ethyl acetate, nitrocellulose, toluene sulphonamide formaldehyde resin, dibutyl phthgalate, toluene, styrene, and acetone. In one embodiment, the composition includes from about 50 to about 95 percent, by weight, of the carrier, such as from about 70 to about 90 percent, by weight.

Colorant

In one embodiment, the invention featiures a method for coloring a nail by applying to the nail a composition of the present invention further containing a colorant. What is meant by a colorant is an ingredient which imparts color to the nail. Examples of colorants include, but are not limited to, pigments, dyes, and titanated micas such as the following: titanium dioxide; FD&C and D&C colors such as D&C Red 33: carmine; lake colorants such as red 40 lake and blue 2 lake, ferrocyanide, iron oxides, and ultramarines. In one embodiment, the composition includes from about 0.01 to about 30 percent, by weight, of at least one colorant, such as from about 0.1 to about 5 percent, by weight.

Clay

In one embodiment, the composition further contains a clay, for example, to aid in the suspension of the colorant. Examples of clays include, but are not limited to, stearalalkonium hectorite, and bentonite. In one embodiment, the composition includes from about 0.01 to about 20 percent, by weight, of at least one clay, such as from about 0.1 to about 2 percent, by weight.

Additional Active Agents

In one embodiment, the topical composition further contains an active agent. What is meant by an “active agent” is a compound (e.g., a synthetic compound or a compound isolated from a natural source) that has a cosmetic or therapeutic effect on the nails, including, but not limiting to, anti-microbial agents, anti-fungal agents, anti-inflammatory agents, anti-mycotic agents, anti-parasite agents, external analgesics, antioxidants, keratolytic agents, moisturizers, nutrients, vitamins, energy enhancers, firming agents, and agents for nail conditioning.

In one embodiment, the agent is selected from, but not limited to, the group consisting of panthenol, phytantriol, anti-fungal agents such a azoles (e.g., miconazole, econazole, itraconazole, sertaconazole, and ketoconazole), carotenoids, free radical scavengers, spin traps, retinoids such as retinol and retinyl palmitate, ceramides, polyunsaturated fatty acids, essential fatty acids, enzymes, enzyme inhibitors, minerals, coenzyme Q10, amino acids such a proline, vitamins, lactobionic acid, acetyl-coenzyme A, niacin, biotin, proteins, riboflavin, thiamin, ribose, electron transporters such as NADH and FADH2, sunscreens, and other botanical extracts such as aloe vera, and derivatives and mixtures thereof. The active agent will typically be present in the composition of the invention in an amount of from about 0.001% to about 20% by weight of the composition, e.g., about 0.005% to about 10% such as about 0.01% to about 5%.

Examples of vitamins include, but are not limited to, vitamin A, vitamin Bs such as vitamin B3, vitamin B5, and vitamin B12, vitamin C, vitamin K, and vitamin E and derivatives thereof.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, and propolis.

Other Materials

Various other materials may also be present in the compositions useful in the subject invention. These include humectants, proteins and polypeptides, chelating agents (e.g., EDTA), and preservatives (e.g., parabens), and pH adjusting agents. In addition, the topical compositions useful herein can contain conventional cosmetic adjuvants, such as dyes, opacifiers (e.g., titanium dioxide), pigments, and fragrances.

Mineral Water

The compositions of the present invention may be prepared using a mineral water, for example mineral water that has been naturally mineralized such as Evian® Mineral Water (Evian, France). In one embodiment, the mineral water has a mineralization of at least about 200 mg/L (e.g., from about 300 mg/L to about 1000 mg/L). In one embodiment, the mineral water contains at least about 10 mg/L of calcium and/or at least about 5 mg/L of magnesium.

Percent Film Retention Test

The “percent film retention” of compositions of the present invention can be determined by the following assay. An adjustable pipette is used to apply approximately 0.5 ml of the composition to three microscope slides (3 inch long by 1 inch wide are used for sample preparation) from left to right leaving 3 cm of uncoated area on the right end of each slide. Prior to application of the composition, an initial tare weight of the slides are obtained. Following application of the composition, the slides are dried in a fume hood for 24 hours. After drying the slides, they are weighed (“Initial Gross Weight”) and the amount of composition on the slide is calculated by subtracting the tare weight of the slide from the Initial Gross Weight of the slide (“Initial Net Weight”). A small binder clip is then attached to the uncoated end of the slide.

A 30% w/w Sodium Lauryl Ether Sulfate solution in deionized water is prepared, and 1500 ml of this solution is added to a 2000 ml glass beaker having a diameter of about 13.5 cm (the “SLES Bath”). The SLES Bath is placed on a hot plate (Corning Inc., Model PC-620), mixed with slow propeller agitation using a mixer (IKA Mixer, Model# RW20DZM.nS1 Wilmington, North Carolina), and heated to 40-45° C. This temperature is maintained throughout the test. The slides are then completely submerged in the SLES Bath by sliding a 0.3×20 cm wooden rod through the binder clips on each slide and hanging the slides into the SLES Bath. Upon emersion of the slides, the SLES Bath is agitated with propeller agitation at 550-600 RPM. The propeller is positioned so as to not come in contact with the slides. One slide is removed at 30 minutes (to determine the film retention time at 30 minutes), 60 minutes (to determine the film retention time at 30 minutes), and 90 minutes (to determine the film retention time at 30 minutes) following submersion and rinsed with deionized water.

The slides are then placed in a 50° C. incubator (Precision Scientific, Winchester Virginia) for 30 minutes. The slides are then removed from the incubator and dried further for 24 hours at room temperature. The slides are then weighed (“Final Gross Weight”) and the amount of composition remaining on the slide is calculated by subtracting the tare weight of the slide from the Final Gross Weight of the slide (“Final Net Weight”). The percent film retention is calculated by the following formula: (Final Net Weight)/(Initial Net Weight)*100%.

In one embodiment, the composition of the present invention have a percent film retention at 30 minutes of from about 95% to about 50% (such as from about 90% to about 75%), at 60 minutes of from about 85% to about 50% (such as from about 80% to about 75%), and/or a percent film retention at 90 minutes of from about 80% to about 50% (such as from about 75% to about 60%). A benefit of a composition having such a percent film retention is that the composition will remain on the nail upon getting wet but can be removed by washing without the need to a harsh solvent such as acetone.

The composition and formulations containing such compositions of the present invention may be prepared using methodology that is well known by an artisan of ordinary skill.

### EXAMPLE 1

The following is a description of the manufacture of a composition according to the present invention.

TABLE I

| Trade Name | INCI Name | % (w/w) | Supplier |
|---|---|---|---|
| Ethanol | SD 40B Alcohol | 87.85 | AAPER Corp., |
| | | | Shelbyville, KY |
| Silicones Plus | Polysilicone-8 | 2 | 3M, St Paul MN |
| Polymer VS80 | | | |
| dl-Panthenol | Panthenol | 2 | Rita Corporation, |
| | | | Woodstock IL |
| Avalure AC 315 | Acrylates | 7 | Noveon Cleveland, |
| | Copolymer | | OH |
| Vitamin E Acetate | Tocopheryl | 0.25 | BASF Corp. Mount |
| | Acetate | | Olive, NJ |
| Phytantriol | Phytantriol | 0.25 | BASF Corp. Mount |
| | | | Olive, NJ |
| Butylene Glycol | Butylene | 0.1 | Celanese Pasedena, |
| | Glycol | | TX |
| Neo Heliopan BB | Benzophenone-3 | 0.1 | Haarmon & Reimer |
| | | | Teterboro, NJ |
| Calcium Chloride | Calcium | 0.1 | Spectrum Chemical, |
| | Chloride | | Gardena CA |
| Fragrance | Fragrance | 0.35 | IFF, New York, NY |

First, the alcohol was transferred into a tank and agitated using a high-speed air mixer. The polysilicone-8 was then slowly added to the alcohol and mixed for 5 minutes. The panthenol was then added and mixed for 5 minutes. The acrylates copolymer was then slowly added while using high speed and sweep action mixing and allowed to mix for 60 minutes. During such continued mixing, the tocopheryl acetate, phytantriol, butylene glycol, benzophenone-3, and calcium chloride were added and mixed until all are completely dissolved. Then, the fragrance was added and mixed for 10 minutes. The resulting composition was then re-circulated for 15 minutes through a 400 mesh fabric and filtered back into the tank.

### EXAMPLE 2

Compositions 2A-2I of Table 2 were manufactured using the process of Example 1.

TABLE 2

| INCI Name | 2A | 2B | 2C | 2D | 2F | 2G | 2H | 2I |
|---|---|---|---|---|---|---|---|---|
| Ethanol | — | — | 87 | — | — | — | — | — |
| SD Alcohol 40B | — | — | — | 84.5 | 75.63 | — | 86.5 | — |
| SD Alcohol 40-2 | 87 | 82 | — | — | — | — | — | 86.17 |
| Isopropyl Alcohol | — | — | — | — | — | 87.5 | — | — |
| Acrylates Copolymer | 10 | 15 | 10 | 12.5 | 21.87 | 10 | 11.25 | 11.25 |
| Polysilicone-8 | — | — | — | — | — | — | — | 0.3 |
| Triethanolamine | — | — | — | — | — | — | — | 0.03 |
| Tocopheryl Acetate | 0.5 | 0.5 | 0.5 | 0.5 | 0.25 | 0.25 | 0.25 | 0.25 |
| Panthenol | — | — | — | — | — | 2 | 2 | 2 |
| Pantothenic Acid | 2 | 2 | 2 | 2 | 2 | — | — | — |
| Phytantriol | 0.5 | 0.5 | 0.5 | 0.5 | 0.25 | 0.25 | 0.25 | 0.25 |

### EXAMPLE 3

The composition of Table 3 was manufactured in the following manner. The alcohol was added into a container followed by the addition of panthenol, acrylates copolymer, polysilione-8, tocopheryl acetate, and phytantriol. The ingredients were mix until completely dissolved. Then, the calcium chloride and benzophenone-3 were added, and the mixture was mixed well.

TABLE 3

| Trade Name | INCI Name | % w/w | Supplier |
|---|---|---|---|
| Ethanol | SD 40B Alcohol | 70.199 | AAPER Corp., |
| | | | Shelbyville, KY |
| dl Panthenol | Panthenol | 2 | RITA Corp. |
| | | | Woodstock LI |
| Avalure AC 315 | Acrylates | 24 | Noveon, Cleveland |
| | copolymer | | OH |
| Silicone Plus | Polysilicone-8 | 0.6 | 3M, St Paul |
| Polymer VS 80 | | | Minnesota |
| Vitamin E Acetate | Tocopheryl | 0.25 | BASF Corp. Mount |
| | Acetate | | Olive, NJ |
| Phytantriol | Phytantriol | 0.25 | BASF Corp. Mount |
| | | | Olive, NJ |
| Calcium Chloride | Calcium | 0.1 | Spectrum Chemical, |
| | Chloride | | Gardena CA |
| Neo Heliopan BB | Benzophenone-3 | 0.3 | Haarmon & Reimer, |
| | | | Teterboro NJ |
| Butylene Glycol | Butylene | 1 | Celanese Pasadena, |
| | Glycol | | TX |
| Red 40 Lake | Red 40 Lake | 0.45 | Warner Jenkinson, |
| | | | South Plainfield NJ |
| Blue 2 Lake | Blue 2 Lake | 0.037 | Warner Jenkinson, |
| | | | South Plainfield NJ |
| Titanium Dioxide | Titanium | 0.264 | Whitaker Clark & |
| 328 | Dioxide | | Daniels, South |
| | | | Plainfield NJ |
| Mastergel PCX- | Stearalkonium | 0.2 | Sud Chemie., |
| ETH-6029 | Bentonite | | Louisville, KY |
| Sparkling Citrus | Fragrance | 0.35 | IFF New York, NY |

### EXAMPLE 4

Compositions 4A-4E of Table 4 were manufactured using the process of Example 3.

TABLE 4

| INCI Name | 4A | 4B | 4C | 4D | 4E |
|---|---|---|---|---|---|
| SD 40B Alcohol | — | 73.63 | 79.75 | 81.92 | 72.468 |
| SD Alcohol 40-2 | 81.4 | — | — | — | — |
| Acrylates Copolymer | 10 | 21.87 | 13 | 13 | 20 |
| Polysilicone-8 | — | — | — | 0.3 | 1 |
| Acrylates/VA | 3 | — | 3 | 0.5 | 1.5 |
| Crosspolymer | | | | | |
| Trethanolamine | — | — | — | 0.03 | 0.03 |
| PEG-15 Cocomonium | 0.6 | — | — | — | — |
| Chloride | | | | | |
| Tocopheryl Acetate | 0.5 | 0.25 | 0.25 | 0.25 | 0.25 |
| Panthenol | — | 2 | 1.75 | 1.75 | 1.75 |
| Pantothenic Acid | 2 | — | — | — | — |
| Phytantriol | 0.5 | 0.25 | — | 0.25 | 0.25 |
| Calcium Chloride | — | — | — | | 0.75 |
| Mica and Titanium | 2 | — | — | — | — |
| Dioxide | | | | | |
| Mica and Titanium | — | 2 | 2 | 2 | — |
| Dioxide | | | | | |
| Bismuth Oxychloride | — | — | — | — | 0.195 |
| D&C Red 33 | — | — | — | — | 0.001 |
| Mica and Titanium | — | — | — | — | 0.511 |
| Dioxide and Carmine | | | | | |
| Mica and Titanium | — | — | — | — | 1.17 |
| Dioxide | | | | | |
| Mica and Titanium | — | — | — | — | 0.125 |
| Dioxide and Carmine and | | | | | |
| Ferric Ferrocyanide | | | | | |

### EXAMPLE 5

The percent film retention of the composition of Example 1 and Example 3 were compared to a standard anhydrous nail lacquer (OPI Nail Lacquer Nagellack, OPI Products Inc., North Hollywood, Calif.), and the water-based nail polishes Barbie® Totally Nails (Tara Toy Corporation, Hauppauge, N.Y.) and Dumais TC-E11-MC. The film retentions at 30 minutes, 60 minutes, and 90 minutes are set forth below in Table 5.

TABLE 5

| | 30 mins. | 60 mins. | 90 mins. |
|---|---|---|---|
| OPI Nail Lacquer | 97.51 | 97.14 | 96.66 |
| Example 1 | 88.64 | 80.37 | 71.47 |
| Example 3 | 88.87 | 79.42 | 67.93 |
| Barbie ® Totally Nails | 41.49 | 30.96 | 26.72 |
| Dumais TC-E11-MC | 43.12 | 35.6 | 17.87 |

As depicted in Table 5, almost all of the anhydrous nail lacquer remained on the slide, even after 90 minutes of washing. On the other hand, less than 50 percent of both of the water-based products remained on the slides in the first 30 minutes and less than 25 percent remained after 90 minutes. The water-based compositions of the present invention, however, lost only about 12 percent following 30 minutes and only about 30 percent after ninety minutes, thus indicating the compositions would withstand being wet, but yet would be removable with washing.

### EXAMPLE 6

The following study was performed to determine the in vitro nail penetration rate of [¹⁴C]-panthenol from the composition of Example 1, wherein panthenol was replaced with a mixture of 1.90% panthenol and 0.10% [¹⁴C]-panthenol to a total panthenol concentration of 2%, (“Test Composition”). [¹⁴C]-panthenol was obtained from American Radiolabled Chemicals, Inc. (St. Louis, Mo., USA). Healthy human finger nails collected from cadavers were used in the study. The study was divided into two groups. One group of nails received the Test Composition and one group of nails received a mixture of 1.89% panthenol and 0.11% [¹⁴C]-panthenol to a total panthenol concentration of 2% in distilled water (“Control Composition”). Prior to application of the above compositions, the nails were washed with saline, and re-hydrated by placing them in a cloth wetted with saline for three hours.

Aliquots (15 μL) of either the Test Composition or the Control Composition were applied daily to the surface of the nail plate. After seven days of dosing, the inner ventral section of the nail plate was assayed for absorbed content of [¹⁴C]-panthenol. The results indicated that after weight normalization (i.e., the amount of panthenol was divided by the weight of the drilled nail material it was extracted from) that [¹⁴C]-panthenol equivalent in the ventral/intermediate nail plate center for the nails receiving the Test Composition was almost two-fold higher than that of the nails receiving the Control Composition (p<0.05). In addition, the amount of [¹⁴C]-panthenol penetrating through the nails receiving the Test Composition was two times higher than the amount of [¹⁴C]-panthenol penetrating through the nails receiving the Control Composition (p<0.05).

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A method for administering an active agent into the nail, said method comprising applying to said nail a composition comprising (i) an active agent, (ii) at least one film-forming polymer and (iii) a carrier comprising water, alcohol, or a mixture thereof, wherein said composition has a percent film retention at 30 minutes of from about 95% to about 50%.

2. A method of claim 1, wherein said active agent is selected from the group consisting of panthenol, an anti-fungal agent, vitamins, enzymes, and minerals.

3. A method of claim 1, wherein said active agent is selected from the group consisting of panthenol and ketoconazole.

4. A method of claim 1, wherein said composition has a percent film retention at 90 minutes of from about 80% to about 50%.

5. A method of claim 2, wherein said composition has a percent film retention at 90 minutes of from about 80% to about 50%.

6. A method of claim 3, wherein said composition has a percent film retention at 90 minutes of from about 80% to about 50%.

7. A method of claim 1, wherein said at least one film-forming polymer comprises an acrylate polymer.

8. A method of claim 3, wherein said at least one film-forming polymer comprises an acrylate polymer.

9. A method of claim 6, wherein said at least one film-forming polymer comprises an acrylate polymer.

10. A method of claim 7, wherein said at least one film-forming polymer further comprises a polysilicone-polyacryl copolymer.

11. A method of claim 8, wherein said at least one film-forming polymer further comprises a polysilicone-polyacryl copolymer.

12. A method of claim 9, wherein said at least one film-forming polymer further comprises a polysilicone-polyacryl copolymer.

13. A method for strengthening the nail, said method comprising applying to said nail a composition comprising (i) panthenol, (ii) at least one film-forming polymer, and (iii) a carrier comprising water, alcohol, or a mixture thereof, wherein said composition has a percent film retention at 30 minutes of from about 95% to about 50%.

14. A method of claim 13, wherein said composition has a percent film retention at 90 minutes of from about 80% to about 50%.

15. A method of claim 13, wherein said at least one film-forming polymer comprises an acrylate polymer.

16. A method of claim 14, wherein said at least one film-forming polymer comprises an acrylate polymer.

17. A method of claim 15, wherein said at least one film-forming polymer further comprises a polysilicone-polyacryl copolymer.

18. A method of claim 16, wherein said at least one film-forming polymer further comprises a polysilicone-polyacryl copolymer.

19. A composition suitable for application as a nail polish, said composition comprising (i) an active agent, (ii) at least one film-forming polymer, and (iii) a carrier comprising water, alcohol, or a mixture thereof, wherein said composition has a percent film retention at 30 minutes of from about 95% to about 50%.

20. A composition of claim 19, wherein said active agent is selected from the group consisting of panthenol, an anti-fungal agent, vitamins, enzymes, and minerals.

21. A composition of claim 19, wherein said active agent is panthenol.

22. A composition of claim 19, wherein said composition has a percent film retention at 90 minutes of from about 80% to about 50%.

23. A composition of claim 21, wherein said composition has a percent film retention at 90 minutes of from about 80% to about 50%.

24. A composition of claim 19, wherein said at least one film-forming polymer comprises an acrylate polymer.

25. A composition of claim 21, wherein said at least one film-forming polymer comprises an acrylate polymer.

26. A composition of claim 22, wherein said at least one film-forming polymer comprises an acrylate polymer.

27. A composition of claim 23, wherein said at least one film-forming polymer comprises an acrylate polymer.

28. A composition of claim 24, wherein said at least one film-forming polymer further comprises a polysilicone-polyacryl copolymer.

29. A composition of claim 25, wherein said at least one film-forming polymer further comprises a polysilicone-polyacryl copolymer.

30. A composition of claim 26, wherein said at least one film-forming polymer further comprises a polysilicone-polyacryl copolymer.

31. A composition of claim 27, wherein said at least one film-forming polymer further comprises a polysilicone-polyacryl copolymer.
